(19) United States
(12) Patent Application Publication
(10) Pub. No.: US 2026/0102067 A1
(43) Pub. Date: Apr. 16, 2026

(54) APPARATUS AND METHOD FOR 24-HOUR AMBULATORY BLOOD PRESSURE MONITORING WITH COMPENSATION BASED ON POSTURE AND/OR ACTIVITY DETECTION

(71) Applicant: CARDIAI TECHNOLOGIES LTD., Calgary (CA)

(72) Inventors: Anmol KAPOOR, Calgary (CA); Sidharth Singh BHINDER, Calgary (CA)

(21) Appl. No.: 19/114,083

(22) PCT Filed: Sep. 22, 2023
(86) PCT No.: PCT/CA2023/051258

§ 371 Date: Mar. 21, 2025

Related U.S. Application Data
(60) Provisional application No. 63/408,887, filed on Sep. 22, 2022.

(51) Int. Cl. A61B 5/021; A61B 5/00; A61B 5/0205; A61B 5/11

(52) U.S. Cl. CPC A61B 5/021; A61B 5/02055; A61B 5/1118; A61B 5/4561; A61B 5/6833; A61B 5/7264

(57) ABSTRACT

An apparatus for monitoring a person's health conditions. The apparatus has one or more biometric sensors for periodically measuring the person's biometric data, one or more activity sensors for periodically measuring the person's activities, and a circuit functionally coupled to the one or more biometric sensors and the one or more activity sensors, the circuit having an artificial intelligence (AI) engine configured for using a trained AI model for analyzing the person's health condition based on the measurement data collected from the one or more biometric sensors and the one or more activity sensors.

### CROSS-REFERENCE TO RELATED APPLICATIONS

This application claims the benefit of U.S. Provisional Patent Application Ser. No. 63/408,887, filed Sep. 22, 2022, the content of which is incorporated herein by reference in its entirety.

### FIELD OF THE DISCLOSURE

The present disclosure relates generally to blood-pressure monitoring, and in particular to 24-hour ambulatory blood-pressure monitoring with compensation based on posture and/or activity detection.

### BACKGROUND

Blood pressure is closely related to a person's health condition. High blood pressure (also called “hypertension”) or low blood pressure (also called “hypotension”) may cause various health issues. However, a one-time blood-pressure measurement conducted in a doctor's office or clinical setting may not be sufficient to confirm hypertension or hypotension, and 24-hour ambulatory blood pressure (24 hr ABP) monitoring is often required, wherein the blood pressure of a person or patient is periodically (e.g., every 20 to 30 minutes during the day and every hour at night) or continuously monitored for at least 24 hours with the heart rate being monitored at the same time.

### SUMMARY

According to one aspect of this disclosure, there is provided an apparatus for monitoring a person's health conditions, the apparatus comprising: one or more biometric sensors for periodically measuring the person's biometric data; one or more activity sensors for periodically measuring the person's activities; and a circuit functionally coupled to the one or more biometric sensors and the one or more activity sensors, the circuit comprising an artificial intelligence (AI) engine configured for: using a trained AI model for analyzing the person's health condition based on the measurement data collected from the one or more biometric sensors and the one or more activity sensors.

In some embodiments, the one or more biometric sensors and the one or more activity sensors are configured to periodically conduct a set of measurements for periodically measuring the person's biometric data and the person's activities; and the one or more activity sensors are configured for: in conducting each set of measurements, measuring the person's activities at least two of before, during, and after the one or more biometric sensors measure the person's biometric data.

In some embodiments, the apparatus further comprises: one or more stickable pads for sticking to the person's body; each of the one or more stickable pads comprises at least one sensor of the one or more biometric sensors and the one or more activity sensors.

In some embodiments, said using a trained AI model for analyzing the person's health condition comprises: determining, based on the measurement data collected by the one or more biometric sensors and the one or more activity sensors, whether any of the one or more biometric sensors and the one or more activity sensors is erroneous; and if the one or more biometric sensors and the one or more activity sensors are not erroneous, analyzing the person's health condition based on the measurement data collected by the one or more biometric sensors and the one or more activity sensors.

In some embodiments, the person's activities comprise at least one of the person's movement, and the person's postures.

In some embodiments, the one or more biometric sensors comprises at least one sensor of one or more blood-pressure sensors and one or more heart-rate sensors, and the person's biometric data comprises the person's blood-pressure data and the person's heart-rate data.

In some embodiments, the one or more biometric sensors further comprises at least one sensor of one or more temperature sensors for measuring the person's body temperatures, one or more oxygen saturation sensors for measuring the person's oxygen levels, and one or more vibration sensors or one or more microphones for measuring sound from the person's body.

According to one aspect of this disclosure, there is provided a computerized method for monitoring a person's health conditions, the method comprising: periodically measuring the person's biometric data using one or more biometric sensors; periodically measuring the person's activities using one or more activity sensors; and using a trained AI model for analyzing the person's health condition based on the measurement data collected from the one or more biometric sensors and the one or more activity sensors.

In some embodiments, said periodically measuring the person's biometric data and periodically measuring the person's activities comprise: periodically conducting a set of measurements using one or more biometric sensors and one or more activity sensors for periodically measuring the person's biometric data and the person's activities, respectively; and, in conducting each set of measurements, measuring the person's activities using the one or more activity sensors at least two of before, during, and after the one or more biometric sensors measure the person's biometric data.

In some embodiments, the computerized method further comprises: sticking one or more stickable pads to the person's body; each of the one or more stickable pads comprises at least one sensor of the one or more biometric sensors and the one or more activity sensors.

In some embodiments, said using a trained AI model for analyzing the person's health condition comprises: determining, based on the measurement data collected by the one or more biometric sensors and the one or more activity sensors, whether any of the one or more biometric sensors and the one or more activity sensors is erroneous; and if the one or more biometric sensors and the one or more activity sensors are not erroneous, analyzing the person's health condition based on the measurement data collected by the one or more biometric sensors and the one or more activity sensors.

In some embodiments, the person's activities comprise at least one of the person's movement, and the person's postures.

In some embodiments, the one or more biometric sensors comprises at least one sensor of one or more blood-pressure sensors and one or more heart-rate sensors, and the person's biometric data comprises the person's blood-pressure data and the person's heart-rate data.

In some embodiments, the one or more biometric sensors further comprises at least one sensor of one or more temperature sensors for measuring the person's body temperatures, one or more oxygen saturation sensors for measuring the person's oxygen levels, and one or more vibration sensors or one or more microphones for measuring sound from the person's body.

According to one aspect of this disclosure, there is provided one or more non-transitory computer-readable storage devices comprising computer-executable instructions, wherein the instructions, when executed, cause one or more circuits to actions comprising: periodically measuring the person's biometric data using one or more biometric sensors; periodically measuring the person's activities using one or more activity sensors; and using a trained AI model for analyzing the person's health condition based on the measurement data collected from the one or more biometric sensors and the one or more activity sensors.

In some embodiments, said periodically measuring the person's biometric data and periodically measuring the person's activities comprise: periodically conducting a set of measurements using one or more biometric sensors and one or more activity sensors for periodically measuring the person's biometric data and the person's activities, respectively; and, in conducting each set of measurements, measuring the person's activities using the one or more activity sensors at least two of before, during, and after the one or more biometric sensors measure the person's biometric data.

In some embodiments, the actions further comprising: sticking one or more stickable pads to the person's body; each of the one or more stickable pads comprises at least one sensor of the one or more biometric sensors and the one or more activity sensors.

In some embodiments, said using a trained AI model for analyzing the person's health condition comprises: determining, based on the measurement data collected by the one or more biometric sensors and the one or more activity sensors, whether any of the one or more biometric sensors and the one or more activity sensors is erroneous; and if the one or more biometric sensors and the one or more activity sensors are not erroneous, analyzing the person's health condition based on the measurement data collected by the one or more biometric sensors and the one or more activity sensors.

In some embodiments, the person's activities comprise at least one of the person's movement, and the person's postures.

In some embodiments, the one or more biometric sensors comprises at least one sensor of one or more blood-pressure sensors and one or more heart-rate sensors, and the person's biometric data comprises the person's blood-pressure data and the person's heart-rate data.

In some embodiments, the one or more biometric sensors further comprises at least one sensor of one or more temperature sensors for measuring the person's body temperatures, one or more oxygen saturation sensors for measuring the person's oxygen levels, and one or more vibration sensors or one or more microphones for measuring sound from the person's body.

### BRIEF DESCRIPTION OF THE DRAWINGS

For a more complete understanding of the disclosure, reference is made to the following description and accompanying drawings, in which:

FIG. 1 is a schematic diagram of a health monitoring and analysis system according to some embodiments of this disclosure;

FIG. 2 is a schematic diagram showing a simplified hardware structure of the client computing device and the server computer of the health monitoring and analysis system shown in FIG. 1, according to some embodiments of this disclosure;

FIG. 3 is a schematic diagram showing a simplified software structure of the client computing device and the server computer of the health monitoring and analysis system shown in FIG. 1, according to some embodiments of this disclosure;

FIG. 4 is a schematic diagram showing a simplified hardware structure of the health measurement device of the health monitoring and analysis system shown in FIG. 1, according to some embodiments of this disclosure;

FIG. 5 is a schematic diagram showing a function structure of the health monitoring and analysis system shown in FIG. 1, according to some embodiments of this disclosure;

FIG. 6 is a flowchart showing the steps of a blood-pressure monitoring and analysis procedure performed by the health monitoring and analysis system shown in FIG. 1, according to some embodiments of this disclosure;

FIG. 7 is a schematic diagram of a health monitoring and analysis system according to some other embodiments of this disclosure;

FIG. 8 is a schematic diagram of a health monitoring and analysis system according to yet some other embodiments of this disclosure;

FIG. 9 is a schematic diagram showing a health measurement device of the health monitoring and analysis system shown in FIG. 1, according to some embodiments of this disclosure;

FIG. 10 is a schematic cross-sectional view of a pad of the health measurement device shown in FIG. 9, according to some embodiments of this disclosure;

FIG. 11 is a schematic diagram showing a blood-pressure sensor coupled to a pad shown in FIG. 10, according to some embodiments of this disclosure;

FIG. 12 is a schematic diagram showing a health measurement device of the health monitoring and analysis system shown in FIG. 1, according to some embodiments of this disclosure;

FIG. 13 is a schematic diagram showing a health measurement device of the health monitoring and analysis system shown in FIG. 1, according to yet some embodiments of this disclosure; and

FIG. 14 is a schematic diagram showing a health measurement device of the health monitoring and analysis system shown in FIG. 1, according to still some embodiments of this disclosure; and

FIG. 15 is a schematic diagram showing a health measurement device of the health monitoring and analysis system shown in FIG. 1, according to yet still some embodiments of this disclosure.

### DETAILED DESCRIPTION

Embodiments disclosed herein relate to blood-pressure monitoring, and more specifically, relate to 24-hour ambulatory blood pressure (24 hr ABP) monitoring.

Generally, blood-pressure readings (or simply “blood pressure”) comprise (1) systolic blood pressure which is the blood pressure when the heart beats, and (2) diastolic blood pressure which is the blood pressure when the heart is resting between beats. The blood-pressure is often denoted as “(systolic blood pressure reading)/(diastolic blood pressure reading)”. For example the blood pressure of 120/80 millimeters of mercury (mm Hg) means that the systolic blood pressure reading is 120 mm Hg and the diastolic blood pressure is 80 mm Hg.

The normal blood-pressure values of a healthy person are lower than 120 mm Hg and lower than 80 mm Hg, respectively.

If a person's systolic blood pressure is between 120 mm Hg and 129 mmHg, and the diastolic blood pressure is below 80 mm Hg, the person may have a risky health condition of an elevated blood pressure.

If the systolic blood pressure is between 130 mm Hg and 139 mmHg, or the diastolic blood pressure is between 80 mm Hg and 89 mm Hg, the person may be diagnosed with hypertension stage 1.

If the systolic blood pressure is greater than 140 mm Hg, or the diastolic blood pressure is greater than 90 mm Hg, the person may be diagnosed with hypertension stage 2.

If the systolic blood pressure is greater than 180 mm Hg, and/or the diastolic blood pressure is greater than 120 mm Hg, the person may be diagnosed with hypertensive crisis.

Hypertension stage 1, hypertension stage 2, and hypertensive crisis are often collectively called “hypertension” or “high blood pressure”.

On the other hand, too-low blood pressure may be diagnosed with “hypotension” which usually includes absolution hypotension and orthostatic hypotension. If the resting blood-pressure (that is, the blood pressure when the person is resting) is below 90/60 mmHg, the person may be diagnosed with absolution hypotension. If the blood pressure drops within three minutes after the person stands up from a sitting position with the systolic blood pressure dropping more than 20 mmHg and the diastolic blood pressure dropping more than 10 mmHg, the person may be diagnosed with orthostatic hypotension (also called “postural hypotension”).

As those skilled in the art understand, blood pressure is generally related to the person's posture and activity. Moreover, a one-time blood pressure measurement conducted in a doctor's office or clinical setting may not be sufficient to confirm hypertension or hypotension. Therefore, 24-hour ambulatory blood pressure (24 hr ABP) monitoring is often used, wherein the blood pressure of a person or patient is periodically (for example, every 20 to 30 minutes during the day and every hour at night) or continuously monitored for at least 24 hours with the heart rate being monitored at the same time.

With the blood-pressure data collected during a 24 Hr ABP, a blood-pressure profile for the person in their usual environment without the stresses that the person may otherwise experience in clinical settings. Such a blood-pressure profile may allow accurate blood-pressure analysis and the diagnosis of blood-pressure related health issues.

As those skilled in the art understand, a person's blood pressure may change or vary in accordance with the person's daily activities and sleep patterns. Therefore, the analysis of 24 Hr ABP measurements may need to take into account the person's daily activities and sleep patterns.

According to one aspect of this disclosure, a health monitoring and analysis system is disclosed. The health monitoring and analysis system comprises a health measurement device attachable to a person. The health measurement device comprises a plurality of sensors such as one or more biometric sensors (such as one or more blood-pressure sensor 202 and/or one or more heart-rate sensors 204, oxygen saturation sensors, CO₂ saturation sensors, temperature sensors, vibration sensors, other audio, optical, thermal, pressure sensors, an/or the like) for periodically measuring the person's biometric data (such as blood pressure, heart rate, respiratory rate, pulse rate, electrocardiogram (ECG), photoplethysmography (PPG), oxygen level, CO₂ level, breathing sound, body mass index (BM)/weight/height, and/or the like), and one or more activity sensors. The health measurement device uses the plurality of sensors to collect measurement data of the person's blood pressure, heart rate, posture, and activities, and is in communication with a client computing device and/or a server computer for using an artificial intelligence (AI) engine and a trained AI model for analyzing the person's health conditions based on the collected measurement data.

In some embodiments, the health measurement device provides a user-friendly interface and device settings allowing patients and doctors to operate with simple instructions. When the health measurement device is carried by or otherwise attached to a person (such as a patient), the health measurement device may measure and monitor a person's blood pressure, heart rate, and other information at regular intervals (for example, every 20 to 30 minutes during the day and every hour at night) for an extended time such as 48 hours. In some embodiments, the frequency and timing of measurements may be easily programmed.

In some embodiments, the activity sensors may collect posture and activity measurement data during the time intervals when the blood-pressure sensor and heart-rate sensor are collecting their measurement data).

In some other embodiments, the activity sensors may collect posture and activity measurement data (such as acceleration and angular velocity) before, during, and/or after the time intervals when the blood-pressure sensor and heart-rate sensor are collecting their measurement data). For example, the activity sensors may collect posture and activity measurement data once before each blood-pressure and/or hear-rate measurement, approximately 10 to 15 times during each measurement, and once after each measurement (thus giving rise to approximately 12 to 17 sensor datasets for each blood-pressure and/or hear-rate measurement), which may be used to identify the person's activity (such as upright, walking, running, resting, sleeping, or the like) and create working diagrams for more accurately analyzing the blood-pressure and/or heart-rate measurement data.

In yet some other embodiments, the activity sensors may continuously collect posture and activity measurement data during the entire 24 hr ABP monitoring period (or a longer ABP monitoring period as needed).

The measurement data may be stored in the health measurement device, in the client computing device, and/or in the server computer (such as in the person's personal cloud account).

The collected measurement data thus include the person's blood-pressure data, heart-rate data, posture data, and activity data throughout the person's everyday activities including sleep. More specifically, the collected measurement data may be categorized into:
blood-pressure measurement data when the person is active;
heart-rate measurement data when the person is active;
blood-pressure measurement data when the person is at rest; and
heart-rate measurement data when the person is at rest.

By analyzing the collected measurement data, the health monitoring and analysis system may provide accurate reports of the person's health measurements such as the person's blood pressure status, hear rate status, posture profile, activity profile (for example, active or at rest), and/or the like.

By using the AI engine and the trained AI model in the analysis of the person's health conditions, the health monitoring and analysis system may accurately identify activity-caused and/or posture-caused fluctuation in the person's blood-pressure and/or heart rate. For example, the health monitoring and analysis system may accurately identify that the person's blood-pressure increase during night was caused by the person getting up. As another example, the health monitoring and analysis system may accurately identify that the person's high blood pressure during sleeping at night was caused by the person's snoring. The health monitoring and analysis system may also accurately identify the person's blood-pressure fluctuation caused by the change of the person's posture (for example, from laying down to standing up).

By using the AI engine and the trained AI model in the analysis of the person's health conditions, the health monitoring and analysis system may also identify sensor errors that may cause irregular data samples in the collected blood-pressure and/or heart-rate measurement data.

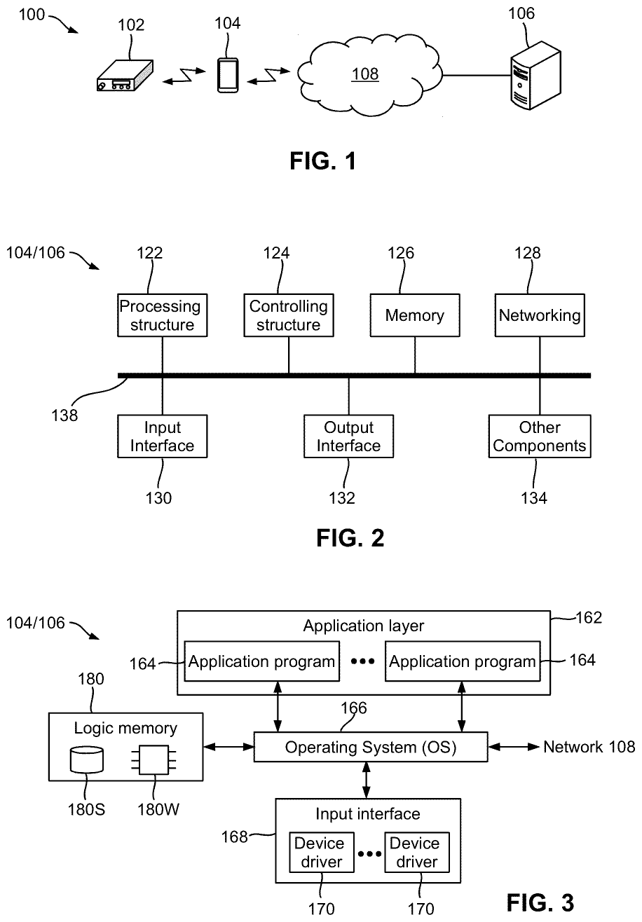

Turning now to FIG. 1, a health monitoring and analysis system according to some embodiments of this disclosure is shown and is generally identified using reference numeral 100. The health monitoring and analysis system 100 comprises a health measurement device 102 attachable to a person. The health measurement device 102 is in wireless communication with a client computing device 104 via a suitable wireless or wired communication technology such as BLUETOOTH® (BLUETOOTH is a registered trademark of Bluetooth Sig Inc., Kirkland, WA, USA), Bluetooth Low Energy (BLE), WI-FI® (WI-FI is a registered trademark of Wi-Fi Alliance, Austin, TX, USA), Ethernet, Z-Wave, Long Range (LoRa), ZIGBEE® (ZIGBEE is a registered trademark of ZigBee Alliance Corp., San Ramon, CA, USA), wireless broadband communication technologies such as Global System for Mobile Communications (GSM), Code Division Multiple Access (CDMA), Universal Mobile Telecommunications System (UMTS), Worldwide Interoperability for Microwave Access (WiMAX), CDMA2000, Long Term Evolution (LTE), 3GPP, the fifth-generation New Radio (5G NR), the six generation (6G) wireless network, and/or the like. The client computing device 104 is in wireless or wired communication with one or more server computers 106 via a network 108.

In these embodiments, the health measurement device 102 is used for measuring and monitoring a person's blood pressure such as for 24 Hr ABP monitoring, and monitoring the person's daily activities and sleep patterns. The health measurement device 102 reports the measured data of the person's blood-pressure, activities, and sleep patterns to the client computing device 104. The client computing device 104 uses the data received from the health measurement device 102 to analyze the person's health conditions, and may display the analysis results on a screen thereof. The client computing device 104 may use the services provided by the server computer 106 for facilitating its health-condition analysis and/or store the data received from the health measurement device 102 and the analysis results to the server computer 106.

The client computing device 104 may be a portable or non-portable computing device such as a smartphone, a tablet, a personal digital assistant (PDA), a laptop computer, a desktop computer, and/or the like.

The server computer 106 may be a computing device designed specifically for use as a server, or a general-purpose computing device acting as a server computer while also being used by various users.

Generally, the client computing device 104 and the server computer 106 have a similar hardware structure such as the hardware structure shown in FIG. 2. As shown, the computing device 104/106 comprises a processing structure 122, a controlling structure 124, one or more non-transitory computer-readable memory or storage devices 126, a network interface 128, an input interface 130, and an output interface 132, functionally interconnected by a system bus 138. The computing device 104/106 may also comprise other components 134 coupled to the system bus 138.

The processing structure 122 may be one or more single-core or multiple-core computing processors such as INTEL® microprocessors (INTEL is a registered trademark of Intel Corp., Santa Clara, CA, USA), AMD® microprocessors (AMD is a registered trademark of Advanced Micro Devices Inc., Sunnyvale, CA, USA), ARM® microprocessors (ARM is a registered trademark of Arm Ltd., Cambridge, UK) manufactured by a variety of manufactures such as Qualcomm of San Diego, California, USA, under the ARM® architecture, or the like. When the processing structure 122 comprises a plurality of processors, the processors thereof may collaborate via a specialized circuit such as a specialized bus or via the system bus 138.

The processing structure 122 may also comprise one or more real-time processors, programmable logic controllers (PLCs), microcontroller units (MCUs), μ-controllers (UCs), specialized/customized processors and/or controllers using, for example, field-programmable gate array (FPGA) or application-specific integrated circuit (ASIC) technologies, and/or the like.

Generally, each processor of the processing structure 122 comprises necessary circuitries implemented using technologies such as electrical and/or optical hardware components for executing one or more processes as the implementation purpose and/or the use case maybe, to perform various tasks. In many embodiments, the one or more processes may be implemented as firmware and/or software stored in the memory 126. Those skilled in the art will appreciate that, in these embodiments, the one or more processors of the processing structure 122, are usually of no use without meaningful firmware and/or software.

Of course, those skilled the art will appreciate that a processor may be implemented using other technologies such as analog technologies.

The controlling structure 124 comprises one or more controlling circuits, such as graphic controllers, input/output chipsets, and the like, for coordinating operations of various hardware components and modules of the computing device 104/106.

The memory 126 comprises one or more one or more non-transitory computer-readable storage devices or media accessible by the processing structure 122 and the controlling structure 124 for reading and/or storing instructions for the processing structure 122 to execute, and for reading and/or storing data, including input data and data generated by the processing structure 122 and the controlling structure 124. The memory 126 may be volatile and/or non-volatile, non-removable or removable memory such as RAM, ROM, EEPROM, solid-state memory, hard disks, CD, DVD, flash memory, or the like. In use, the memory 126 is generally divided into a plurality of portions for different use purposes. For example, a portion of the memory 126 (denoted as storage memory herein) may be used for long-term data storing, for example, for storing files or databases. Another portion of the memory 126 may be used as the system memory for storing data during processing (denoted as working memory herein).

The network interface 128 comprises one or more network modules for connecting to other computing devices or networks through the network 108 by using suitable wired and/or wireless communication technologies. In some embodiments, parallel ports, serial ports, USB connections, optical connections, or the like may also be used for connecting other computing devices or networks although they are usually considered as input/output interfaces for connecting input/output devices.

The input interface 130 comprises one or more input modules for one or more users to input data via, for example, touch-sensitive screens, touch-sensitive whiteboards, touch-pads, keyboards, computer nice, trackballs, microphones, scanners, cameras, and/or the like. The input interface 130 may be a physically integrated part of the computing device 104/106 (for example, the touch-pad of a laptop computer or the touch-sensitive screen of a tablet), or may be a device physically separated from but functionally coupled to, other components of the computing device 104/106 (for example, a computer mouse). The input interface 130, in some implementation, may be integrated with a display output to form a touch-sensitive screen or a touch-sensitive whiteboard.

The output interface 132 comprises one or more output modules for output data to a user. Examples of the output modules include displays (such as monitors, LCD displays, LED displays, projectors, and the like), speakers, printers, virtual reality (VR) headsets, augmented reality (AR) goggles, and/or the like. The output interface 132 may be a physically integrated part of the computing device 104/106 (for example, the display of a laptop computer or a tablet), or may be a device physically separate from but functionally coupled to other components of the computing device 104/106 (for example, the monitor of a desktop computer).

The computing device 104/106 may also comprise other components 134 such as one or more positioning modules, temperature sensors, barometers, inertial measurement units (IMUs), and/or the like. Examples of the positioning modules may be one or more global navigation satellite system (GNSS) components (for example, one or more components for operation with the Global Positioning System (GPS) of USA, Global'naya Navigatsionnaya Sputnikovaya Sistema (GLONASS) of Russia, the Galileo positioning system of the European Union, and/or the Beidou system of China).

The system bus 138 interconnects various components 122 to 134 enabling them to transmit and receive data and control signals to and from each other.

FIG. 3 shows a simplified software structure of the computing device 104 or 106. The software structure 160 comprises an application layer 162, an operating system 166, a logical input/output (I/O) interface 168, and a logical memory 172. The application layer 162, operating system 166, and logical I/O interface 168 are generally implemented as computer-executable instructions or code in the form of software programs or firmware programs stored in the logical memory 172 which may be executed by the processing structure 122.

The application layer 162 comprises one or more application programs 164 executed by or performed by the processing structure 122 for performing various tasks.

The operating system 166 manages various hardware components of the computing device 104 or 106 via the logical I/O interface 168, manages the logical memory 172, and manages and supports the application programs 164. The operating system 166 is also in communication with other computing devices (not shown) via the network 108 to allow the application programs 164 to communicate with programs running on other computing devices. As those skilled in the art will appreciate, the operating system 166 may be any suitable operating system such as MICROSOFT® WINDOWS® (MICROSOFT and WINDOWS are registered trademarks of the Microsoft Corp., Redmond, WA, USA), APPLE® OS X, APPLE® iOS (APPLE is a registered trademark of Apple Inc., Cupertino, CA, USA), Linux, ANDROID® (ANDROID is a registered trademark of Google Inc., Mountain View, CA, USA), or the like. The computing devices 104 or 106 of the computer network system 100 may all have the same operating system, or may have different operating systems.

The logical I/O interface 168 comprises one or more device drivers 170 for communicating with respective input and output interfaces 130 and 132 for receiving data therefrom and sending data thereto. Received data may be sent to the application layer 162 for being processed by one or more application programs 164. Data generated by the application programs 164 may be sent to the logical I/O interface 168 for outputting to various output devices (via the output interface 132).

The logical memory 172 is a logical mapping of the physical memory 126 for facilitating the application programs 164 to access. In this embodiment, the logical memory 172 comprises a storage memory area that may be mapped to a non-volatile physical memory such as hard disks, solid-state disks, flash drives, and/or the like, generally for long-term data storage therein. The logical memory 172 also comprises a working memory area that is generally mapped to high-speed, and in some implementations, volatile physical memory such as RAM, generally for application programs 164 to temporarily store data during program execution. For example, an application program 164 may load data from the storage memory area into the working memory area, and may store data generated during its execution into the working memory area. The application program 164 may also store some data into the storage memory area as required or in response to a user's command.

In a server computer 106, the application layer 162 generally comprises one or more server-side application programs 164 which provide(s) server functions for managing network communication with the client computing device 104 (as well as other computing devices connect to the network 108) and facilitating collaboration between the server computer 106 and the client computing device 104. Herein, the term “server” may refer to a server computer 106 from a hardware point of view, or to a logical server from a software point of view, depending on the context.

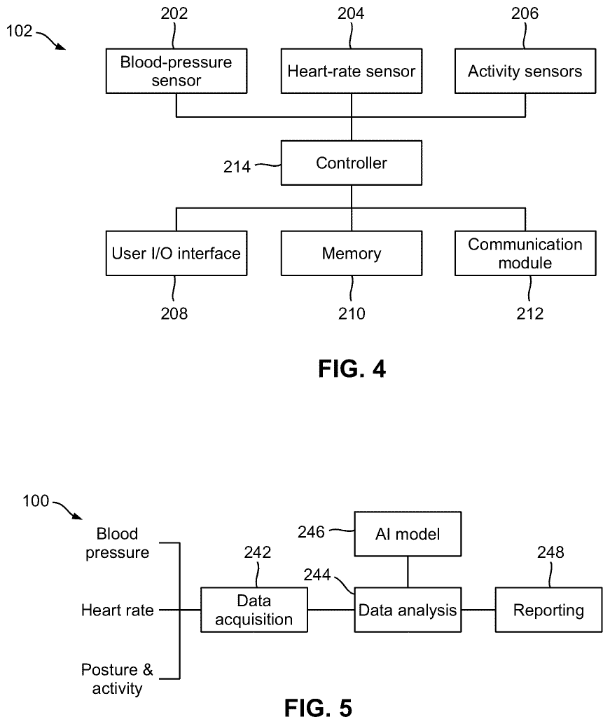

In these embodiments, the health measurement device 102 is a small, portable machine for measuring a person's blood pressure, heart rate, and other information at regular intervals for an extended time such as 48 hours. FIG. 4 is a schematic diagram showing the hardware structure of health measurement device 102, according to some embodiments of this disclosure.

As shown, the health measurement device 102 comprises one or more biometric sensors for periodically measuring the person's biometric data (such as one or more blood-pressure sensor 202 and/or one or more heart-rate sensors 204 for periodically measuring the person's blood-pressure data and heart-rate data), one or more activity sensors 206, a user input/output (I/O) interface 208, a memory 210, and a communication module 212, all connected to a control circuit or controller 214.

The blood-pressure sensor 202 may be in any suitable form such as an upper arm cuff, a finger cuff, a photoplethysmography (PPG) optical sensor for positioning on a wrist, and/or the like. The blood-pressure sensor 202 may use any suitable technology for measuring blood pressure, such as the auscultatory method, the oscillometric technique, ultrasound techniques, the finger cuff method, optical techniques, and/or the like.

The heart-rate sensor 204 may use any suitable technology for measuring the heart rate, such as an electrical sensor measuring heartbeats by detecting electrical signals related to the expansion and contraction of heart chambers (for example, an electrocardiogramsor), an optical sensor for measuring the heart rate by measuring the variation of the blood volume in a blood vessel (for example, a PPG optical sensor), and/or the like.

The activity sensor 206 may be any sensor suitable for detecting the person's posture and activity, such as an accelerometer, a gyroscope, an inertial measurement unit (IMU), an inclinometer, and/or the like.

The user input/output (I/O) interface 208 may comprise any suitable I/O interfacing components for receiving input from a user (such as one or more buttons, a keypad, a keyboard, a computer mouse, a trackball, a touchscreen, a digital pen, a microphone, and/or the like) and for outputting information to the user (such as a screen, a monitor, a light-emitting diode (LED) panel, a speaker, and/or the like).

The memory 210 may be any suitable non-transitory, computer-readable, volatile and/or non-volatile storage devices or media similar to the memory 126 described above.

The communication module 212 may be any suitable module for communicating with the client computing device 104 or other computing devices using wireless and/or wired communication technologies. For example, in some embodiments, the communication module 212 may be a Bluetooth® module.

The controller 214 may be a processing unit or circuit such as an integrated circuit (IC) chip functionally coupled to the modules 202 to 212 for controlling the operation of these modules, receiving measurement data from the blood-pressure sensor 202, the hear-rate sensor 204, and the one or more activity sensors 206, receiving user inputs from the user I/O interface 208, displaying information to the user through the user I/O interface 208, receiving data and/or instructions from other computing devices such as the client computing device 104, and storing various received data and instructions in the memory 210. In some embodiments, the controller 214 may process received data (such as the measurement data received from the blood-pressure sensor 202, the hear-rate sensor 204, and the one or more activity sensors 206), analyze the measurement data, and sending received data and/or analysis results to the client computing device 104. In some other embodiments, the controller 214 may not analyze the measurement data. Rather, the analysis of the measurement data may be conducted by the client computing device 104 and/or the server computer 106.

FIG. 5 is a schematic diagram showing the function structure of the health monitoring and analysis system 100 according to some embodiments of this disclosure. As shown, the health monitoring and analysis system 100 comprises a data acquisition module 242 for measurement data acquisition, a data analysis module 244 having an artificial intelligence (AI) engine (such as a machine-learning engine) for analyzing the acquired data using a trained AI model 246, and a reporting module 248 for reporting the analysis results. In these embodiments, the AI model 246 may be any suitable AI model such as a machine learning model, a deep neural network model, a clustering model, a convolutional neural network, or the like, and may be trained using historical measurement data collected from a plurality of people.

Herein, the data acquisition module 242 is generally implemented in the health measurement device 102. In some embodiments, the data analysis module 244, the AI model 246, and the reporting module 248 are implemented in the client computing device 104. In some other embodiments, some of the data analysis module 244, the AI model 246, and the reporting module 248 may be implemented in the server computer 106.

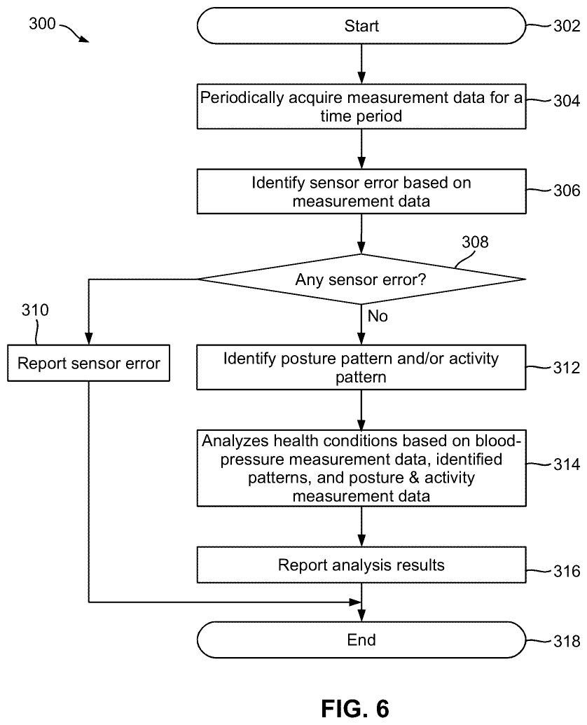

FIG. 6 is a flowchart showing the steps of a blood-pressure monitoring and analysis procedure 300 performed by the health monitoring and analysis system 100.

When the procedure 300 starts (step 302), the health monitoring and analysis system 100 uses the data acquisition module 242 to acquire measurement data of blood pressure, heart rate, and the person's posture and activity from the blood-pressure sensor 202, the heart-rate sensor 204, and the one or more activity sensors 206 (step 304). The acquired measurement data is then sent to the data analysis module 244 for analysis.

At step 306, the data analysis module 244 uses the AI engine and the trained AI model 246 to identify sensor errors based on the acquired measurement data. As those skilled in the art will appreciate, a person's blood pressure and heart rate have a generally corresponding relationship. For example, a high heart rate generally corresponds to a high blood pressure. Moreover, the person's blood pressure and heart rate also have a generally corresponding relationship with the person's posture and activity. For example, a person in an intensive activity usually experience high heart rate and high blood pressure.

Those skilled in the art will also appreciate that such relationships are generally complicated, and instant measurements of the person's blood pressure and/or heart rate may not in consistent with such relationships and appear to be biased therefrom. There exist various reasons that may cause such measurement biases, such as due to so-called “noise”, data error (for example, caused by sensor errors), a sudden change in the person's health conditions, and/or the like. Thus, one needs to correctly identify the reasons causing the measurement biases and take corresponding actions. For example, if the cause of the measurement bias is due to the noise, one needs to remove the noise and obtain corrected measurement. If the cause of the measurement bias is due to sensor errors, the malfunctioning sensor needs to be replaced. If the cause of the measurement bias is due to a sudden change in the person's health conditions, proper advices (such as visiting a family doctor or going to hospital, making an emergency call, and/or the like) needs to be provided to the person. However, due to the complicated nature of the relationships between the person's blood pressure/heart rate and the person's posture/activity, manually identifying such reasons correctly is impossible or at least very difficult.

By using the trained AI model 246, the AI engine of the data analysis module 244 may identify measurement data samples that are biased from such relationships and determine that the identified measurement data samples are erroneous data samples highly probably caused by malfunction of the sensor that collected these erroneous data samples.

For example, if the blood pressure rises high but the heart rate remain low, either the blood-pressure sensor or the heart-rate sensor may be malfunctioning. Then, if the activity sensor reports that the person is in a highly active situation, then, the blood-pressure sensor is operating correctly and the heart-rate sensor is malfunctioning.

If at step 308, the data analysis module 244 identifies any sensor error, the data analysis module 244 reports the sensor error (step 310) using any suitable methods such as triggering an alarm beep, displaying an error message on the screen of the client computing device 104, sending a message to a user (for example, a caretaker), and/or the like. The procedure 300 then ends (step 318).

If at step 308, the data analysis module 244 does not identify any sensor error, the data analysis module 244 then use the AI engine and the trained AI model 246 to identify the person's posture and/or activity pattern (including sleep pattern) (step 312). For example, the data analysis module 244 may identify that the person is active rather than sleeping when the activity sensor 206 reports the person being in an upright status and the heart-rate sensor 204 reports an increased heart rate. In some embodiments, the data analysis module 244 may identify the person's posture and/or activity pattern as one of upright, walking, running, resting, and sleeping.

At step 314, the data analysis module 244 analyzes the person's health conditions using the AI engine and the trained AI model 246 based on the identified patterns and the measurement data. As described above, the measurement data may be various biometric data such as blood pressure, heart rate, respiratory rate, pulse rate, ECG, PPG, oxygen level, CO2 level, breathing sound, BM/weight/height, and/or the like. Some biometric data may be measured or otherwise obtained from the one or more biometric sensors, some biometric data may be obtained or corrected by collating with relevant other biometric data, some biometric data may be pre-stored biometric data, and some biometric data may be obtained using suitable predictive algorithms such as suitable AI methods.

At step 316, the analysis results are sent from the data analysis module 244 to the reporting module 248 for reporting such as storing the analysis results in a database (such as in the person's personal cloud account), displaying the analysis results to a user, transmitting the analysis results to another computing device such as another client computing device or a server 106, and/or the like. In some embodiments, the reported analysis results may comprise:
activity grading: such as no movement, light movement, or strong movement;
posture report: such as upright, laying down, sleeping, and/or the like;
blood-pressure grading: such as hypotension, normal, or hypertension; and
heart-rate grading: high, normal, or low.

In some embodiments, if the data analysis module 244 determines that the blood-pressure, heart-rate, posture, and activity measurement data are not properly correlated and no sensor error is identified, the data analysis module 244 may instruct the reporting module 248 to report a risk of heart diseases or failure and trigger an alarm to the person.

After reporting, the procedure 300 then ends (step 318).

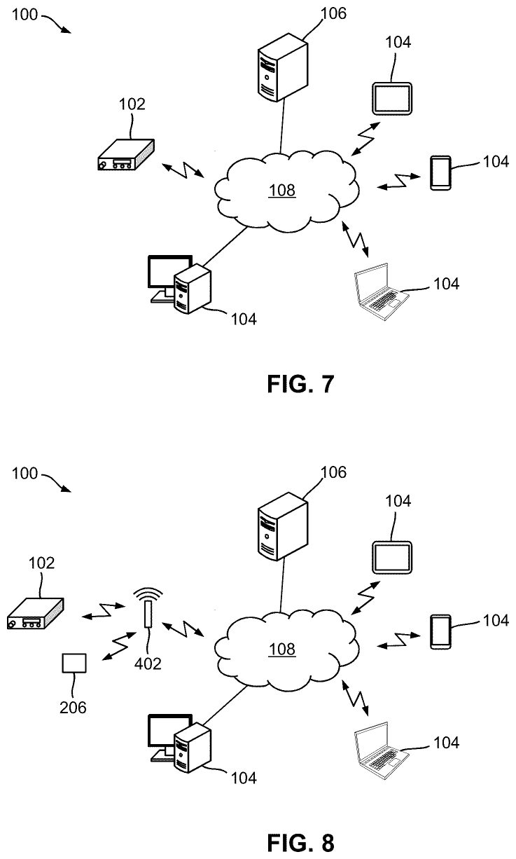

Those skilled in the art will appreciate that various embodiments are readily available. For example, as shown in FIG. 7, in some embodiments, the health monitoring and analysis system 100 may comprise a plurality of client computing devices 104, and the health measurement device 102 may be directly connected to the network 108 via suitable wireless and/or wired communication technologies.

In some other embodiments as shown in FIG. 8, some sensors such as the activity sensors 206 may not be included in the health measurement device 102. Rather, these sensors may be implemented as separate sensor devices. In these embodiments, the health measurement device 102 and the activity sensors 206 may be connected to the network 108 via one or more access points 402.

In some embodiments, the health measurement device 102 may not comprise any heart-rate sensor 204 and thus does not monitor the person's heart rates.

In some embodiments, the health measurement device 102 may not monitor the person's postures, and thus the activity sensor 206 is only for monitoring the person's movements.

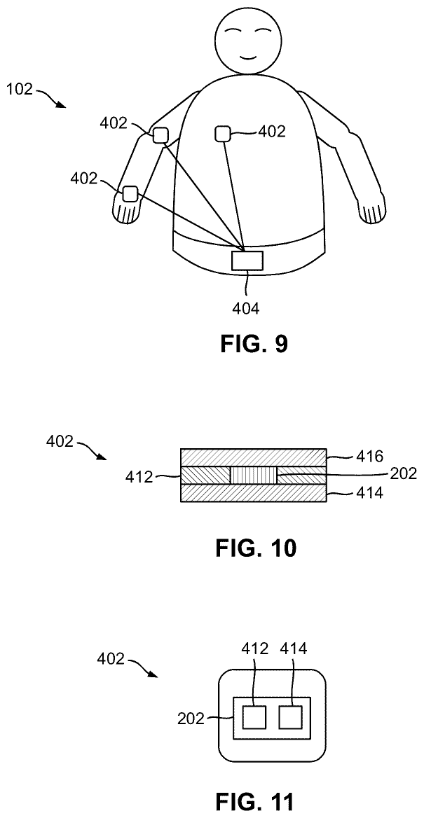

In some embodiments as shown in FIG. 9, the health measurement device 102 may comprise one or more stickable pads 402 for sticking to suitable positions of a user, such as the user's chest, wrist, elbow area, ankle area, and/or the like. Each pad 402 comprises a blood-pressure sensor 202 which is connected to a central unit 404 attached to, for example, a belt on the user. In these embodiments, the central unit 404 may comprise other modules such as the activity sensors 206, user I/O interface 208, memory 210, communication module 212, and controller 214. In these embodiments, the heart-rate sensor 204 may be a separate component attachable to the user (such as a separate pad suitable for sticking to the user's body).

FIG. 10 is a schematic cross-sectional view of a pad 402, according to some embodiments of this disclosure. As shown, the pad 402 comprise a sensor layer 412 having a blood-pressure sensor 202, an attachment layer 414 coupled to a front side of the sensor layer 412 and having a suitable sticky material for sticking to a person's skin, and a protection layer 416 coupled to a rear side of the sensor layer 412.

FIG. 11 is a schematic diagram showing a blood-pressure sensor 202 (coupled to a pad 402), according to some embodiments of this disclosure. As shown, the blood-pressure sensor 202 comprises a light emitter 412 such as a pulse light-emitting diode and a light sensor 414. When the blood-pressure sensor 202 is coupled to a pad 402, the attachment layer 414 may comprise a transparent sticky material and/or may comprise an opening at a position corresponding to that of the blood-pressure sensor 202, to allow light passing through the attachment layer 414.

In operation, the light emitter 412 emits two light beams of two different wavelengths towards the user's skin. The light beams are reflected by the peripheral artery under the user's skin and are captured by the light sensor 414. Then, the controller 214 may use photoplethysmography (PPG) to analyze the captured light beams to obtain the user's blood pressure.

In some embodiments, each activity sensor 206 may be in the form of a stickable pad.

In some embodiments, at least one pad 402 may comprise two or more of a blood-pressure sensor 202, a heart-rate sensor 204, and an activity sensor 206.

In above embodiments, the one or more blood-pressure sensors 202 and one or more heart-rate sensors 204 are used as biometric sensors for periodically measuring the person's biometric data. In some embodiments, the biometric sensors may further comprise one or more temperature sensors for measuring a person's body temperatures at one or more locations, one or more oxygen saturation sensors for measuring the person's oxygen levels, one or more vibration sensors or microphones for measuring sound from various body locations (such as from the person's lung, throat, and/or the like), and/or the like. In some embodiments, a stickable pad may comprise a single one of above-described sensors. In some other embodiments, a stickable pad may comprise a plurality of above-described sensors.

The measured temperature, oxygen level, and sound may be combined with the measured blood pressure and/or hear rates, as well as the measured activities to determine the person's health condition using the AI engine. For example, a risk of heart failure may be determined if the measured activities indicate that the person is walking or running but the measured temperature drops, and/or the oxygen level drops. Similarly, the measured sound may indicate sleep-apnea caused snoring, which, alone or in combination with measured hear rates, may indicate a risk of heart failure. The measured sound may also indicate the status of the lung (for example, fluid filled in the lung which is an indication of risk of cancer), which may be combined with other sensor measurements for more accurate determination of the person's health conditions.

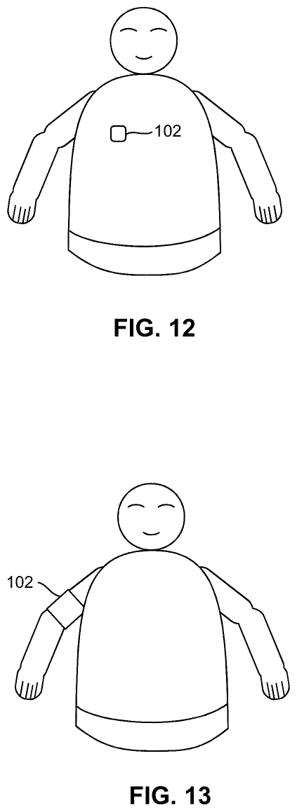

Those skilled in the art will appreciate that the health measurement device 102 may be in any suitable form. For example, FIG. 12 is a schematic diagram showing the health measurement device 102 according to some embodiments of this disclosure. As shown, the health measurement device 102 is in the form of a stickable pad and comprising all components (such as the blood-pressure sensor 202, the heart-rate sensor 204, vibration sensor, controller 214, and the like) therein for sticking to the chest of a person.

In some embodiments as shown in FIG. 13, the health measurement device 102 is in the form of a cuff for positioning around a user's elbow, and comprising all components (such as the blood-pressure sensor 202, the heart-rate sensor 204, controller 214, and the like) therein.

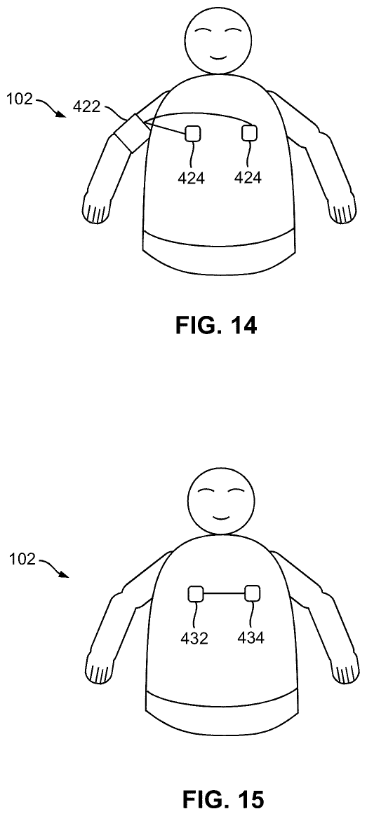

In some embodiments as shown in FIG. 14, the health measurement device 102 comprises a cuff 422 for positioning around a user's elbow, and comprising herein all components except vibration sensor. The health measurement device 102 also comprises a pair of vibration sensors 424 in the form of stickable pads for sticking to chest around the left and right lungs, respectively. The pad 424 is connected to the cuff 422 using a suitable wired or wireless method.

In some embodiments as shown in FIG. 15, the health measurement device 102 comprises a first stickable pad 432 and a second stickable pad 432 for sticking to chest around the left and right lungs, respectively. The first stickable pad 432 comprises all components (such as the blood-pressure sensor 202, the heart-rate sensor 204, controller 214, and the like) therein, and may or may not comprise a vibration sensor. The second stickable pad 434 comprises a vibration sensor and is connected to the first pad 432 using a suitable wired or wireless method.

Although embodiments have been described above with reference to the accompanying drawings, those of skill in the art will appreciate that variations and modifications may be made without departing from the scope thereof as defined by the appended claims.

1. -21. (cancelled)

22. An apparatus for monitoring a person's health conditions, the apparatus comprising:
one or more biometric sensors for periodically measuring the person's biometric data;
one or more activity sensors for periodically measuring the person's activity data; and
a circuit functionally coupled to the one or more biometric sensors and the one or more activity sensors, the circuit comprising an artificial intelligence (AI) engine configured for:
analyzing the person's health condition using a trained AI model based on the biometric data and activity data,
wherein said analyzing the person's health condition comprises:
identifying the person's activities, and
identifying cause of measurement bias based on the identified activities;
wherein said identifying the person's activities comprises, for each biometric data measuring, identifying the person's activity based on:
the person's activity data measured before and during said each biometric data measuring,
the person's activity data measured before and after said each biometric data measuring,
the person's activity data measured during and after said each biometric data measuring, or
the person's activity data measured before, during, and after said each biometric data measuring.

23. The apparatus of claim 22 further comprising:
one or more stickable pads for sticking to the person's body;
wherein each of the one or more stickable pads comprises at least one sensor of the one or more biometric sensors and the one or more activity sensors.

24. The apparatus of claim 22, wherein said using a trained AI model for analyzing the person's health condition comprises:
determining, based on the biometric data and activity data, whether any of the one or more biometric sensors and the one or more activity sensors is erroneous; and
if the one or more biometric sensors and the one or more activity sensors are not erroneous, analyzing the person's health condition based on the biometric data and activity data.

25. The apparatus of claim 22, wherein the person's activities comprise at least one of the person's movement, and the person's postures.

26. The apparatus of claim 22, wherein the one or more biometric sensors comprises at least one sensor of one or more blood-pressure sensors and one or more heart-rate sensors; and
wherein the person's biometric data comprises the person's blood-pressure data and the person's heart-rate data.

27. The apparatus of claim 26, wherein the one or more biometric sensors comprises at least one sensor of one or more temperature sensors for measuring the person's body temperatures, one or more oxygen saturation sensors for measuring the person's oxygen levels, and one or more vibration sensors or one or more microphones for measuring sound from the person's body.

28. A computerized method for monitoring a person's health conditions, the method comprising:
periodically measuring the person's biometric data using one or more biometric sensors;
periodically measuring the person's activity data using one or more activity sensors; and
analyzing the person's health condition using a trained AI model based on the biometric data and activity data;
wherein said analyzing the person's health condition comprises:
identifying the person's activities, and
identifying cause of measurement bias based on the identified activities;
wherein said identifying the person's activities comprises, for each biometric data measuring, identifying the person's activity based on:
the person's activity data measured before and during said each biometric data measuring,
the person's activity data measured before and after said each biometric data measuring,
the person's activity data measured during and after said each biometric data measuring, or
the person's activity data measured before, during, and after said each biometric data measuring.

29. The computerized method of claim 28 further comprising:
sticking one or more stickable pads to the person's body;
wherein each of the one or more stickable pads comprises at least one sensor of the one or more biometric sensors and the one or more activity sensors.

30. The computerized method of claim 28, wherein said using a trained AI model for analyzing the person's health condition comprises:
determining, based on the biometric data and activity data, whether any of the one or more biometric sensors and the one or more activity sensors is erroneous; and
if the one or more biometric sensors and the one or more activity sensors are not erroneous, analyzing the person's health condition based on the biometric data and activity data.

31. The computerized method of claim 28, wherein the person's activities comprise at least one of the person's movement, and the person's postures.

32. The computerized method of claim 28, wherein the one or more biometric sensors comprises at least one sensor of one or more blood-pressure sensors and one or more heart-rate sensors; and
wherein the person's biometric data comprises the person's blood-pressure data and the person's heart-rate data.

33. The computerized method of claim 32, wherein the one or more biometric sensors further comprises at least one sensor of one or more temperature sensors for measuring the person's body temperatures, one or more oxygen saturation sensors for measuring the person's oxygen levels, and one or more vibration sensors or one or more microphones for measuring sound from the person's body.

34. One or more non-transitory computer-readable storage devices comprising computer-executable instructions, wherein the instructions, when executed, cause one or more circuits to actions comprising:
periodically measuring the person's biometric data using one or more biometric sensors;
periodically measuring the person's activity data using one or more activity sensors; and
analyzing the person's health condition using a trained AI model based on the biometric data and activity data;
wherein said analyzing the person's health condition comprises:
identifying the person's activities, and
identifying cause of measurement bias based on the identified activities;
wherein said identifying the person's activities comprises, for each biometric data measuring, identifying the person's activity based on:
the person's activity data measured before and during said each biometric data measuring,
the person's activity data measured before and after said each biometric data measuring,
the person's activity data measured during and after said each biometric data measuring, or
the person's activity data measured before, during, and after said each biometric data measuring.

35. The one or more non-transitory computer-readable storage devices of claim 34, wherein the actions further comprising:
sticking one or more stickable pads to the person's body;
wherein each of the one or more stickable pads comprises at least one sensor of the one or more biometric sensors and the one or more activity sensors.

36. The one or more non-transitory computer-readable storage devices of claim 34, wherein said using a trained AI model for analyzing the person's health condition comprises:
determining, based on the biometric data and activity data, whether any of the one or more biometric sensors and the one or more activity sensors is erroneous; and
if the one or more biometric sensors and the one or more activity sensors are not erroneous, analyzing the person's health condition based on the biometric data and activity data.

37. The one or more non-transitory computer-readable storage devices of claim 34, wherein the person's activities comprise at least one of the person's movement, and the person's postures.

38. The one or more non-transitory computer-readable storage devices of claim 34, wherein the one or more biometric sensors comprises at least one sensor of one or more blood-pressure sensors and one or more heart-rate sensors; and
wherein the person's biometric data comprises the person's blood-pressure data and the person's heart-rate data.

39. The one or more non-transitory computer-readable storage devices of claim 38, wherein the one or more biometric sensors further comprises at least one sensor of one or more temperature sensors for measuring the person's body temperatures, one or more oxygen saturation sensors for measuring the person's oxygen levels, and one or more vibration sensors or one or more microphones for measuring sound from the person's body.
